(19) United States
(12) Patent Application Publication
(10) Pub. No.: US 2024/0041975 A1
(43) Pub. Date: Feb. 8, 2024

(54) ANTICANCER PEPTIDE AND USE THEREOF FOR INHIBITING CANCER CELL VIABILITY

(71) Applicant: NATIONAL TSING HUA UNIVERSITY, HSINCHU (TW)

(72) Inventors: Jya-Wei CHENG, Hsinchu (TW); Chih-Lung WU, Hsinchu (TW); Jing-wen Ding, Hsinchu (TW)

(21) Appl. No.: 18/318,392

(22) Filed: May 16, 2023

Related U.S. Application Data
(62) Division of application No. 17/816,954, filed on Aug. 2, 2022, now abandoned.

(51) Int. Cl. A61K 38/16; A61P 35/00

(52) U.S. Cl. CPC A61K 38/16; A61P 35/00

(57) ABSTRACT

Compositions for peptide therapeutics of cancer are provided. Methods of using such compositions containing the same are also described, and a step of allowing the peptide therapeutics to coexist with a cancer cell to destroy the cancer cell.

### CROSS-REFERENCE TO RELATED APPLICATION

This application is a Divisional Application of the U.S. application Ser. No. 17/816,954, filed Aug. 2, 2022, which is herein incorporated by reference in its entirety

### REFERENCE TO AN ELECTRONIC SEQUENCE LISTING

The contents of the electronic sequence listing (THPA-2021002-US (DA)_SEQUENCELISTING (20230509).xml; Size: 9.21 KB; and Date of Creation: 2023-05-09) is herein incorporated by reference in its entirety.

### FIELD OF THE INVENTION

This disclosure relates to peptide-based therapeutic compositions and methods for inhibiting cancer cell viability and treating cancer.

### BACKGROUND OF THE INVENTION

Cancer is a leading cause of death worldwide. Different types of cancer correspond to various cell types of the body. Cancer cells are characterized by uncontrolled proliferation and the ability to invade surrounding normal tissue or distant sites by homological and/or lymphatic spread.

The difficulty for the effective treatment of cancer relates to establishing the distinction between malignant and normal cells of the body. Both are derived from the same source and are very similar, and for this reason, there is no significant recognition by the immune system as to the threat.

Despite the advances made in cancer therapy over the past few decades, such as surgery, radiotherapy, chemotherapy and immunotherapy, these therapeutic modalities are still associated with significant side effects.

For example, this can be achieved through surgery, but the propensity of the disease to invade adjacent tissues or to spread to distant sites (metastasis) often limits its effectiveness. The chemotherapy effectiveness is, in most cases, limited by its toxicity to other tissues (cells) of the organism, as well as radiotherapy, which can also damage normal tissues. In immunotherapy, carcinogenic cells developed mechanisms to escape from the immune response, a phenomenon known as resistance to treatment.

There is an unmet need for anti-cancer treatments with less side effects and/or for chemotherapy resistant or immunotherapy resistant cancers. The present invention addresses this and other needs.

### SUMMARY OF THE INVENTION

Accordingly, the present invention provides the search for new ways to treat cancer is a process in constant progress, and within this dynamic, new active principles were developed to be useful in the treatment of cancer.

Another aspect of the present invention provides methods for inhibiting or reducing cancer cells by administering to a subject in need thereof a therapeutically effective amount of the peptides.

In summary, these and other objects have been achieved according to the present disclosure which demonstrates the pharmaceutical composition attenuating cancer cells growth, inhibit cancer cells viability, lead to HMGB1 passive release from the cancer cells, lead to release of ATP from the cancer cells, lead to release of cytochrome c from the cancer cells, or/and lead to release of ROS from the cancer cells.

Detailed description of the invention is given in the following embodiments with reference to the accompanying drawings.

### BRIEF DESCRIPTION OF THE DRAWINGS

The drawings described herein are for illustration purposes only and are not intended to limit the scope of the present teachings in any way.

FIG. 1A-1F depict that the cytotoxicity activity of cancer cells via administrating the peptides. FIG. 1A represents the cytotoxicity activity of human non-small cell lung cancer cell A549. FIG. 1B represents the cytotoxicity activity of human non-small cell lung cancer cell PC9. FIG. 1C represents the cytotoxicity activity of human non-small cell lung cancer cell H1975. FIG. 1D represents the cytotoxicity activity of oral squamous cell carcinoma cell OECM-1. FIG. 1E represents the cytotoxicity activity of the oral squamous cell carcinoma cell C9. FIG. 1F represents the cytotoxicity activity of human diploid fibroblast (HFW) as control group.

FIG. 2 depict that cell viability of Human non-small cell lung cancer cells A549 were administrated with 3.226 μM (2 folds of IC₅₀), or 6.452 μM (4 folds of IC₅₀) of K1R12-AGP-RW.

FIG. 3 depict that the release level of HMGB1 was analyzed by Western Blot. Human non-small cell lung cancer cell A549 administrated with 6.452 μM (4×IC₅₀) of K1R12-AGP-RW for different time period (60 min, 120 min, 240 min), the HMGB1 release form lysate (L) to the supernatant (S). A549 treated with medium without serum as control group.

FIG. 4 depict that Detection ATP release into supernatant was detect after adminstrated with K1R12-AGP-RW. Human non-small cell lung cancer cell A549 administrated with 6.452 μM (4×IC₅₀) of K1R12-AGP-RW for different time period (30 min, 60 min, 90 min, 120 min). Data are presented as means ±SEM, ***p<0.005, **p<0.01, versus the control group. Data of FIG. 4 were analyzed using the one-way ANOVA and multiple comparison test.

FIG. 5 depict that release of mitochondria DAMPs Cytochrome c was detected by Elisa kit. Human non-small cell lung cancer cell A549 administrated with 6.452 μM (4×IC₅₀) of K1R12-AGP-RW for different time period (30 min, 60 min, 90 min, 120 min, 240 min). Data are presented as means ±SEM, ***p<0.005, **p<0.01, versus the control group.

FIG. 6 depict that release of ROS from cancer cells were administrated with the peptide. Human non-small cell lung cancer cell A549 administrated with 6.452 μM (4×IC₅₀) of K1R12-AGP-RW for 120 min. The cells of control group were treated with RPMI-1640 without phenol red. Data are presented as means ±SEM, **p<0.05, versus the control group. Data of FIG. 6 were analyzed by Student's t-test.

### DETAILED DESCRIPTION OF THE INVENTION

The following description is merely exemplary in nature and is in no way intended to limit the present teachings, application, of uses.

Optionally, in an exemplary embodiment of the present invention, the peptides of the present invention include, but are not limited to, K1R12 (SEQ ID NO: 1), “IR” represented IKDFR (SEQ ID NO: 2), “RW” represented RRWWRW (SEQ ID NO: 3), “RI” represented RRLVRI (SEQ ID NO: 4), K1R12-AGP-IR (SEQ ID NO: 5), K1R12-AGP-RW (SEQ ID NO: 6), and/or K1R12-AGP-RI (SEQ ID NO: 7).

Sequences of SEQ ID NO: 1-SEQ ID NO: 7 which can be employed in accordance with the invention are shown hereinbelow:
SEQ ID NO: 1:
KRIVQRIKDFLR
SEQ ID NO: 2:
IKDFLR
SEQ ID NO: 3:
RRWWRW
SEQ ID NO: 4:
RRLVRI
SEQ ID NO: 5:
Ac-KRIVQRIKDFLRAGPIKDFLR-N H2
SEQ ID NO: 6:
Ac-KRIVQRIKDFLRAGPRRWWRW-N H2
SEQ ID NO: 7:
Ac-KRIVQRIKDFLRAGPRRLVRI-NH 2

The present invention provides a method for treating, preventing, or alleviating a symptom of cancer by administering to the subject a pharmaceutical composition, optionally in a therapeutically effective amount.

“Subjects” as used herein are generally human subjects and includes, but is not limited to, cancer patients. The subjects may be male or female and may be of any race or ethnicity. The subjects may be of any age, including newborn, neonate, infant, child, adolescent, adult, and geriatric. Subjects may also include animal subjects, particularly mammalian subjects such as canines, felines, bovines, caprines, equines, ovines, porcines, rodents (e.g. mouse, rat, guinea pig, and hamster), lagomorphs, primates (including non-human primates), etc.

The cancers to which the cancer cell inhibitory pharmaceutical compositions of the present invention are applicable are not particularly limited. Examples of the cancers include lung cancer, oral cancer, salivary gland cancer, tongue cancer, pharyngeal cancer, esophagus cancer, stomach cancer, pancreatic cancer, liver cancer, bile duct cancer, duodenal cancer, small intestine cancer, large bowel cancer, colon cancer, rectal cancer, renal cancer, bladder cancer, prostatic cancer, penile cancer, testicular tumor, endometrium cancer, uterine cervix cancer, ovarian cancer, thyroid cancer, parathyroid gland cancer, nasal cavity cancer, paranasal sinus cancer, skin cancer, malignant melanoma, blood cancer, leukemia, malignant lymphoma, myeloma, angiosarcoma, skin cancer, breast cancer or brain tumor.

Further, examples of the lung cancers include large cell lung cancer, squamous cell lung cancer, small cell lung cancer (SCLC) and non-small cell lung cancer (NSCLC).

The cancer cells may be from an established cancer cell line. Alternatively, the cancer cells are primary cancer cells obtained from a cancer subject/cancer patient. In some examples, the cancer cells are lung cancer cells, oral cancer cells, salivary gland cancer cells, tongue cancer cells, pharyngeal cancer cells, esophagus cancer cells, stomach cancer cells, pancreatic cancer cells, liver cancer cells, bile duct cancer cells, duodenal cancer cells, small intestine cancer cells, large bowel cancer cells, colon cancer cells, rectal cancer cells, renal cancer cells, bladder cancer cells, prostatic cancer cells, penile cancer cells, testicular tumor cells, endometrium cancer cells, uterine cervix cancer cells, ovarian cancer cells, thyroid cancer cells, parathyroid gland cancer cells, nasal cavity cancer cells, paranasal sinus cancer cells, skin cancer cells, malignant melanoma cells, blood cancer cells, leukemia cells, malignant lymphoma, myeloma cells, angiosarcoma cells, skin cancer cells, breast cancer cells or brain tumor cells.

In one example, the lung cancer cells include large cell lung cancer cells, squamous cell lung cancer cells, small cell lung cancer (SCLC) cells and non-small cell lung cancer (NSCLC) cells.

A “pharmaceutical composition” is a formulation containing “the anti-cancer peptides” of the present invention in a form suitable for administration to a subject.

When the pharmaceutical composition of the present invention is used as a medicinal drug, various types of dosage forms can be selected depending upon the administration route.

The dose of the pharmaceutical composition of the present invention is appropriately determined depending upon a purpose for therapy or prophylaxis, and conditions such as sexuality, age, weight of a test subject, an administration route, and degree of a disease.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

The data of following EXAMPLES were analyzed and performed by the GraphPad Prism Version. All results of the following EXAMPLES were presented as a mean±SD of at least two independent experiments. ATP secretion and cytochrome c secretion result were using the one-way ANOVA and multiple comparison test. ROS release data were analyzed by Student's t-test. P-value <0.05 were considered statistically significant.

### Example 1

Cell Lines and Culture

Human non-small cell lung cancer line A549, PC9, and H1975 (Osimertinib sensitive; Gefitinib toleran; belonged to T790M mutations), oral squamous cell carcinoma cell line OECM-1 were cultured in RPMI medium supplemented with 10% fetal bovine serum (FBS) and antibiotic. Human oral squamous cell carcinoma cell line C9 and human diploid fibroblast (HFW) were cultured in DMEM medium supplemented with 10% fetal bovine serum (FBS) and antibiotic. Cells were cultured in a humidified incubator containing 5% CO2 at 37° C.

### Example 2

An Anti-Cancer Peptide Preparation

K1R12-AGP-IR (Ac-KRIVQRIKDFLR-AGP-IKDFLR-NH₂) (SEQ ID NO: 5), K1R12-AGP-RW (Ac-KRIVQRIKDFLR-AGP-RRWWRW-NH₂) (SEQ ID NO: 6), K1R12-AGP-RI (Ac-KRIVQRIKDFLR-AGP-RRLVRI-NH₂) (SEQ ID NO: 7) were artificially synthesized. The purity (>95%) of the synthetic peptides was assessed by high-performance liquid chromatography (HPLC) and identity was checked by electrospray mass spectroscopy. The peptide concentration was determined by using the bicinchoninic acid assay (BCA assay).

TABLE 1
The sequence, charge, hydrophobicity and
molecular weight of SEQ ID NO: 5 (KIR12-
AGP-IR), SEQ ID NO: 6 (KIR12-AGP-RW), and
SEQ ID NO: 7 (KIR12-AGP-RI).

| | | | Molecular |
|---|---|---|---|
| | | | Weight |
| SEQ No. | Sequence | Charge | (Da) |
| SEQ ID NO: 5 | Ac-KRIVQRIKDFLR- | +7 | 2611.2 |
| | AGP-IKDFLR-NH₂ | | |
| SEQ ID NO: 6 | Ac-KRIVQRIKDFLR- | +7 | 2865.46 |
| | AGP-RRWWRW-NH₂ | | |
| SEQ ID NO: 7 | Ac-KRIVQRIKDFLR- | +7 | 2632.27 |
| | AGP-RRLVRI-NH₂ | | |

### Example 3

The Cytotoxic Activity of Cancer Cells Via Administrating the Anti-Cancer Peptides from the Present Invention

The cytotoxicity of cancer cells via administrating the peptides were measured by the MTT assay. All cancer cell lines were seeded in a 96-well plate at a density of 5×10⁴ cells per well and incubated for 24 hr. After the medium was removed, using the anti-cancer peptides ranged from 50 μM to 0.78 μM for treatment, diluted in the growth medium, and administered 24 hr. The media was then removed, determined cell viability by incubated for 3 hours at 37 C with the addition of 100 μL Thiazolyl Blue Tetrazolium Bromide (MTT 0.5 mg/ml in PBS; M2128, Sigma-Aldrich) diluted by ratio 1:9 in the growth medium. Next, the medium with MTT solution was removed, added 100 μl Dimethyl Sulfoxide (DMSO, D4540, Sigma-Aldrich) for dissolving the formazan crystal. Cell survival rate was calculated by measuring the absorbance at 570 nm using a microplate reader (Sunrise TM, TECAN, Switzerland). Medium without peptide and mixed with H₂O₂ represented positive and negative control, respectively. The experiments were repeated at least three times. Cell Viability percentage was calculated as follows:

$$Viability ( \% ) = OD \frac{value ( Treatment - Blank )}{ODvalue ( Control - Blank )} \times 100$$

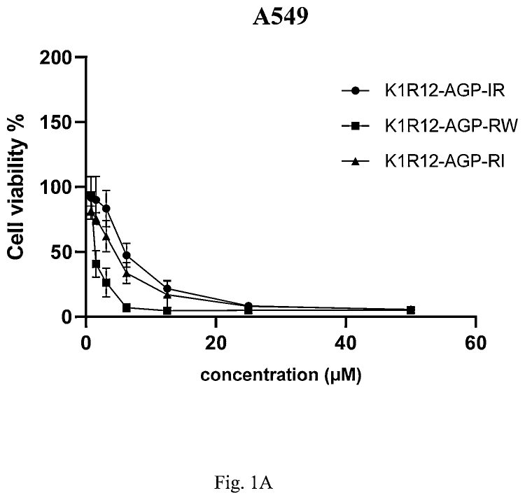

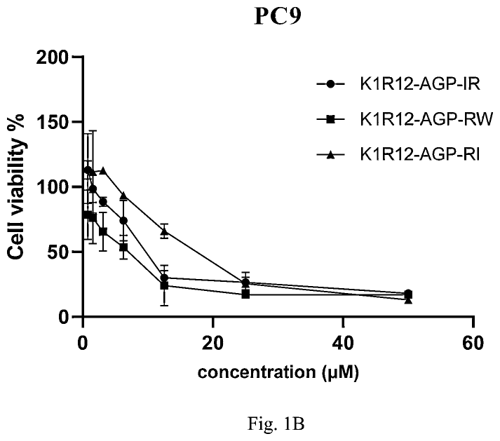

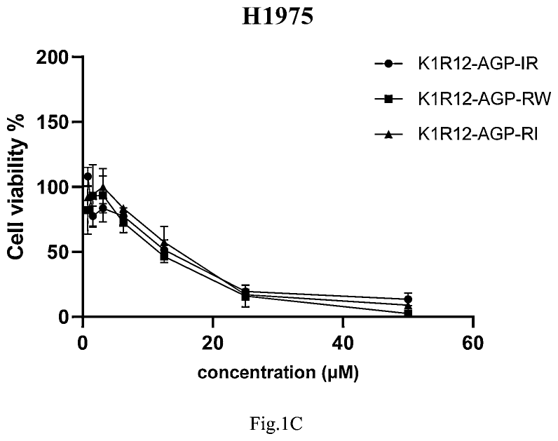

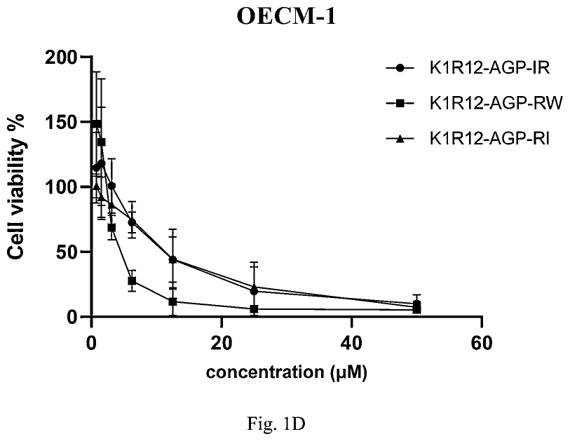

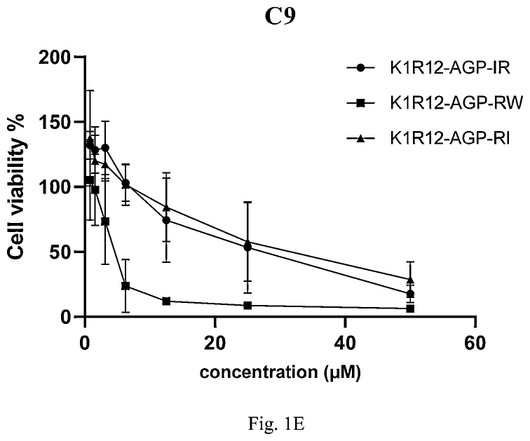

According to the MTT assay result, the K1R12-AGP-IR showed broad-spectrum anticancer activity, against the human lung cancer cell/cell line and oral squamous cell carcinoma cell/cell line (FIG. 1A-FIG. 1F, Table 2-Table 4). K1R12-AGP-IR, K1R12-AGP-RI also showed good efficiency to the human lung cancer cell line (FIG. 1A-FIG. 1C), but weak against the oral squamous cell carcinoma cell line C9 (FIG. 1E). The anti-cancer peptides of K1R12-AGP-RW were more effective to inhibit human non-small cell lung cancer cell line A549 than the other cancer cells, the IC50 of K1R12-AGP-IR, K1R12-AGP-RW, K1R12-AGP-RI was 6.256, 1.613, 3.857 μM respectively (FIG. 1A and Table 2).

TABLE 2
The anticancer activity of human non-small cell lung cancer
line via administrating the anti-cancer peptides.
anticancer activity

| anti-cancer | Human non-small cell lung cancer line | | |
|---|---|---|---|
| peptides | A549 | PC9 | H1975 |
| SEQ ID NO: 5 | 6.256 | 10.49 | 11.88 |
| SEQ ID NO: 6 | 1.613 | 5.418 | 10.90 |
| SEQ ID NO: 7 | 3.857 | 16.82 | 13.71 |

TABLE 3
The anticancer activity of oral squamous cell carcinoma cell
line via administrating the anti-cancer peptides.

| anticancer activity | oral squamous cell | |
|---|---|---|
| anti-cancer | carcinoma cell line | |
| peptides | OECM-1 | C9 |
| SEQ ID NO: 5 | 11.53 | 25.29 |
| SEQ ID NO: 6 | 4.56 | 4.414 |
| SEQ ID NO: 7 | 11.23 | 30.36 |

TABLE 4
The cell cytotoxicity of Normal cell via administrating the
anti-cancer peptides.

| | Cell cytotoxicity |
|---|---|
| | Normal cell |
| anti-cancer peptides | HFW |
| SEQ ID NO: 5 | 13.51 |
| SEQ ID NO: 6 | 11.05 |
| SEQ ID NO: 7 | 10.36 |

### Example 4

Validating Killing Kinetics

To evaluate the cytotoxic activity and time course against the A549 cell, two different concentrations of K1R12-AGP-RW 2×IC50 (3.226 μM), 4×IC50 (6.452 μM) were used. Cells were seeded as described as MTT assay, after 24 hours culture, cell were incubated with 2×IC50 and 4×IC50 peptide solution for 60 minutes, 120 minutes, 180 minutes, 240 minutes. Further incubated in 10% MTT solution for an additional 4 hours. Remove MTT solution and Dimethyl Sulfoxide (DMSO, D4540, Sigma-Aldrich) was added to formazan crystal solubilization. Using a microplate reader (Sunrise TM, TECAN, Switzerland) detecting the absorbance at 570 nm. The experiments were repeated three times independently.

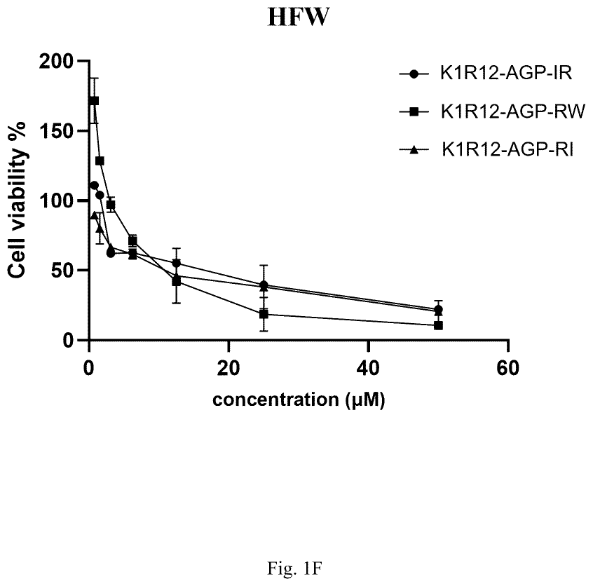

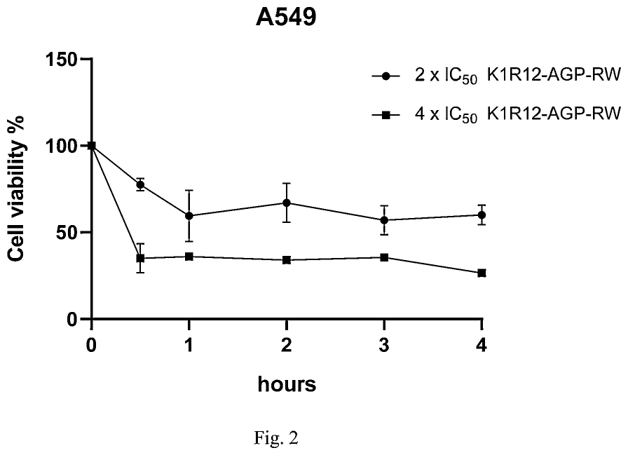

The 4×IC50 (6.452 μM) demonstrated more rapid kill cancer cells within the 240 minutes than 2×IC50 (3.226 μM), and the curve more stable (FIG. 2).

### Example 5

Determinating High Mobility-Group Box-1(HMGB1) Secretion from Cancer Cells Via Administrating the Anti-Cancer Peptides

HMGB1 passive release by necrotic cell, the extracellular HMGB1 will bind several receptors trigger a stronger immune response. But apoptosis cell death induces ROS to release amplify, affect or inactive HMGB1 by oxidation and minimizing immunological effect.

In one embodiment, cancer cells were seeded with 5×10⁵ cells/well in 6-cm polystyrene in complete medium and allowed to adhere 24 hr. Cells were treated with K1R12-AGP-IR, K1R12-AGP-RW, K1R12-AGP-RI at 4×IC50 dilution in medium without serum and incubated at 37° C. for different time points (60 min,120 min,240 min). RPMI medium without peptide treated for 24 hr which was used as a negative control. Supernatants (S) were collected and centrifuged at 1,500 rcf for 5 minutes. Cell lysates (L) use 500 μL Trypsin-EDTA harvested after centrifuged at 750 g for 5 minutes and washing lysate twice with PBS. And addition 150 μL RIPA and protease inhibitor blended buffer, then centrifugation same condition with supernatant(S). Sonication with 5 cycle number, time on or off 30 sec, and centrifugation for 10 minutes, 1400 g at 4° C., thereafter lysed using a 6× Sample buffer, 0.1M DTT (Sigma), and water. Protein concentrations of cell extracts were determined by Bradford reagent. Both supernatants and lysate were boiled in 80° C. 10 minutes and resolved by 12% sodium dodecyl sulfate-polyacrylamide gel electrophoresis (SDS-PAGE), and then electro transferred to a polyvinylidene difluoride (PVDF) membrane (Millipore). The membrane was blocked in 5% skimmed milk and incubated with the HMGB1 antibody (rabbit, polyclonal, Abcam ab 18256). The membrane was then rinsed several times with TB ST, incubated with horseradish peroxidase (HRP)-conjugated secondary antibody (Abcam ab6721). The signals were visualized by enhanced chemiluminescence (ECL) and recorded by a detected system (Image Quant LAS 4000mini).

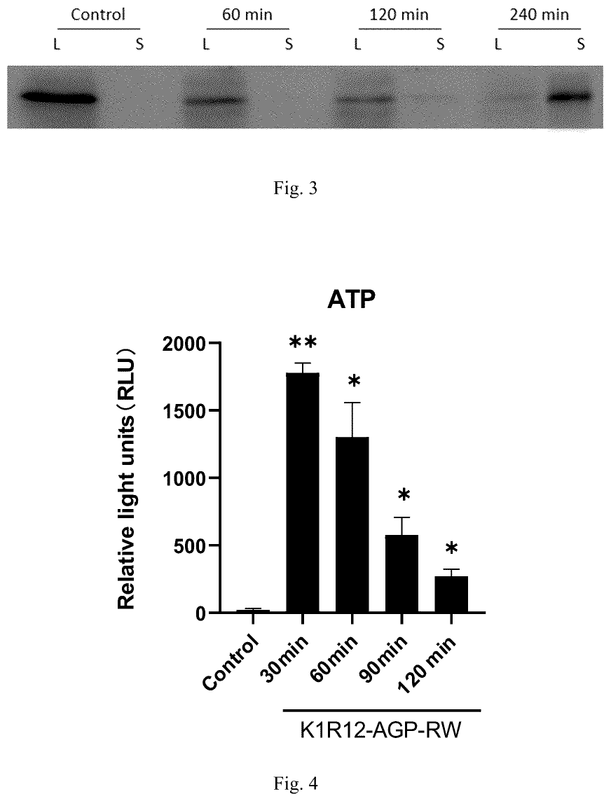

Next, Analyzed treatment with 4×IC50 (6.452 μM) K1R12-AGP-RW after 60, 120, 240 minutes of the HMGB1 release situation by western blot. Time-dependent, HMGB1 exist in cell lysate(L) was reduce. Supernatant(S) HMGB1 slight increase after treatment 120 minutes. After 240 minutes, HMGB1 complete release into the supernatant(S) (FIG. 3).

### Example 6

Cancer Cells Administrated with the Anti-Cancer Peptides Lead to Release of ATP

Dying cell release ATP activates purinergic P2X7 receptor on dendritic cells (DC), further activity the NLRP3-ASC-inflammasome and mediated IL-1β release. And the release of ATP possibly depends on the stages of apoptosis and intracellular stress or cell death promotes.

The ENLITEN ATP assay kit (Promega, Madison, WI, FF2000) was used for the detection of ATP release levels. Above all seeds, cancer cells in 96 well with 1×10⁵ cell per well, incubated 24 hours. On day 2, replace the medium with the fresh medium containing 4×IC₅₀ K1R12-AGP-RW incubated at different time points (30 min, 60 min, 90 min, 120 min). The supernatant was college and centrifuged 500 xg for 10 minutes to remove debris. Add reconstituted rL/L reagent with the sample and detection immediately, expressed as relative light units (RLU). Serially diluted ATP standard to calculating ATP concentrations generates a regression curve. All reagents in this kit must be returned to room temperature before use.

The luciferin-luciferase-based reaction assay was used to test K1R12-AGP-RW treatment A549 extracellular secretion. ATP secretion was demonstrated higher level via administrating with 4×IC₅₀ (6.452 μM) K1R12-AGP-RW treatment within 30 minutes. After that, with time increased, ATP secretion reduced (FIG. 4).

### Example 7

K1R12-AGP-RW Induces Mitochondrial Damage-Associated Molecular Patterns (DAMPs) that Resulted in Extracellular Cytochrome c Release

Cytochrome c is a mitochondrial protein that exists in intermembrane space. Mitochondria outer membrane was perturbed lead to mitochondrial dysfunction, which was affected by the mode of apoptosis cell death. And the more cellular or tissue damage, affect the higher cytochrome c level release. Cytochrome c plays an important role in the respiratory chain of the cell, which function is liable for mitochondrial electron transfer from the cytochrome c reductase complex to the oxidase complex. When mitochondria cytochrome c was depleted, lead to free oxygen radical's generation and decreased production of ATP.

The ELISA kit for detection of cytochrome c was obtained from the R&D System (Minneapolis, MN, USA). Before the experiment, prepare a buffer solution or reagent from the kit. Seed cancer cells 1×10⁵ in 96 well incubated 24 hours. After 24 hours, substitute with 4×IC₅₀ K1R12-AGP-RW dilutions in medium, and incubate designated time points (30 min, 60 min, 90 min, 120 min). Following the supernatant was centrifuged at 16000 xg for 10 minutes. Add 50 μL supernatant and 75 μL of cytochrome c conjugate to each well. Then, add 50 μL of standard, control, or sample per well. Mix by tapping the plate frame for 1 minute. Cover with the adhesive strip provided. Incubate for 2 hours at room temperature. A plate layout is provided to record standards and samples assayed. Then use the wash buffer washed 5 times with 400 μL. The last wash removes any remaining Wash Buffer by decanting. Add 100 μL subtracted reagent reaction 30 minutes at room temperature, that the color change to blue. Detection the cytochrome c at absorbance 450 nm using a microplate reader (Sunrise TM, TECAN, Switzerland).

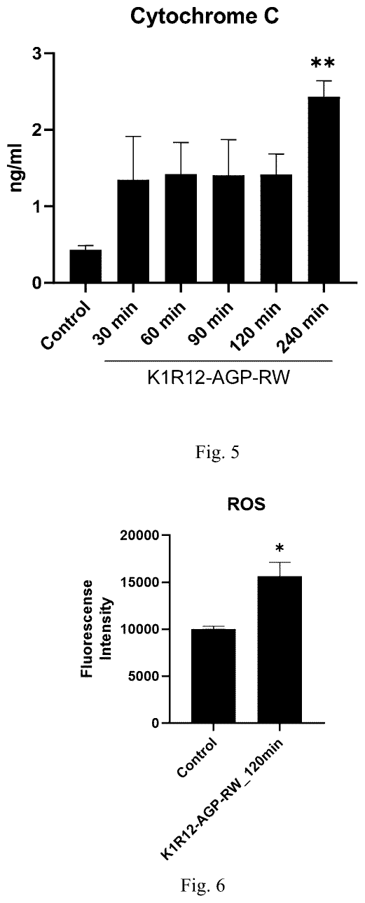

In one embodiment, 4×IC₅₀ (6.452 μM) K1R12-AGP-RW was treat in A549, for 120 minutes, cytochrome c release at a certain level and keep stabilize. Treatment with K1R12-AGP-RW for 240 minutes, cytochrome c has a significant increase than untreated cell (FIG. 5).

### Example 8

ROS Release from Cancer Cells after Administrated with the Anti-Cancer Peptides

Cancer cell growth needs ROS, so a certain level of ROS can promote cancer cell proliferation and progress. When mitochondria were dysfunctional, cytochrome c release increase would enhance ROS production, and promote apoptotic cell.

The 2′,7′-dichlorofluorescin diacetate (DCFDA) intracellular ROS detection assay kit from Abcam (UK, ab113851) was used for measuring ROS release. Cells were seeded in a 96 well black clear bottom plate with 4×10⁴ per well, culture 24 hours. Remove medium and washed once in pre-warm 1× PBS with 100 μl per well. Furthermore, cells were treated with 25 μM DCFDA in 1× buffer solution incubated for 120 minutes. Washed again with a buffer solution of 100 μL/well, before adding the 4×IC₅₀ K1R12-AGP-RW dissolved in RPMI without phenol red, and incubated 60,120 and 240 minutes. Cells incubated with RPMI without phenol red were used as a negative control, and the positive control was tertbutyl hydroperoxide (TBHP). Using a fluorescence plate reader (VICTOR3, PerkinElmer, USA) determined at an excitation wavelength of 485 nm and an emission wavelength of 535 nm.

Treatment with 4×IC₅₀ (6.452 μM) K1R12-AGP-RW for 120 minutes, compared with untreated cell treatment with K1R12-AGP-RW have a significant increase (FIG. 6).

In summary, the present invention demonstrated the anti-cancer peptide, especially K1R12-AGP-RW, administrated with cancer cells resulted in attenuating cancer cells growth, inhibiting cancer cells viability, leading to the cancer cells death, leading to HMGB1 passive release from the cancer cells, leading to release of ATP from the cancer cells, leading to release of cytochrome c from the cancer cells, or/and leading to release of ROS from the cancer cells.

The foregoing is illustrative of the present invention, and is not to be construed as limiting thereof. The invention is defined by the following claims, with equivalents of the claims to be included therein.

What is claimed is:

1. A method for inhibiting cancer cell viability, comprising:
administering to a subject in need thereof a therapeutically effective amount of an anti-cancer peptide, wherein the anti-cancer peptide has a formula, selected from the group consisting of:
K1R12-AGP-C₁,
wherein:
K1R12 is SEQ ID NO: 1;
C₁ is a short peptide of 5-6 amino acids comprising one Arginine (R) residue at least;
wherein C₁ is selected from the group consisting of “IR” (SEQ ID NO: 2), “RW” (SEQ ID NO: 3), or “RI” (SEQ ID NO: 4);
wherein the anti-cancer peptide is both N-terminally acylated and C-terminally amidated;
wherein the anti-cancer peptide is “K1R12-AGP-IR” (SEQ ID NO: 5), “K1R12-AGP-RW” (SEQ ID NO: 6), or “K1R12-AGP-RI” (SEQ ID NO: 7).

2. The method according to claim 1, wherein the cancer cells comprise lung cancer cells, oral cancer cells, salivary gland cancer cells, tongue cancer cells, pharyngeal cancer cells, esophagus cancer cells, stomach cancer cells, pancreatic cancer cells, liver cancer cells, bile duct cancer cells, duodenal cancer cells, small intestine cancer cells, large bowel cancer cells, colon cancer cells, rectal cancer cells, renal cancer cells, bladder cancer cells, prostatic cancer cells, penile cancer cells, testicular tumor cells, endometrium cancer cells, uterine cervix cancer cells, ovarian cancer cells, thyroid cancer cells, parathyroid gland cancer cells, nasal cavity cancer cells, paranasal sinus cancer cells, skin cancer cells, malignant melanoma cells, blood cancer cells, leukemia cells, malignant lymphoma, myeloma cells, angiosarcoma cells, skin cancer cells, breast cancer cells or brain tumor cells.

3. The method according to claim 2, wherein the lung cancer cells are selected from large cell lung cancer cells, squamous cell lung cancer cells, small cell lung cancer (SCLC) cells and non-small cell lung cancer (NSCLC) cells.

4. The method according to claim 2, wherein the anti-cancer peptide can inhibit the cancer cells viability, lead to HMGB1 passive release from the cancer cells, lead to release of ATP from the cancer cells, lead to release of cytochrome c from the cancer cells, or/and lead to release of ROS from the cancer cells.

5. The method according to claim 4, wherein the method comprising the anti-cancer peptide coexist with the cancer cells to destroy the cancer cells.


SEQUENCE LISTING (7 sequences)

SEQ ID NO: 1 - protein, 12 aa, synthetic construct
KRIVQRIKDFLR

SEQ ID NO: 2 - protein, 5 aa, synthetic construct
IKDFR

SEQ ID NO: 3 - protein, 6 aa, synthetic construct
RRWWRW

SEQ ID NO: 4 - protein, 6 aa, synthetic construct
RRLVRI

SEQ ID NO: 5 - protein, 21 aa, synthetic construct
  BINDING 1: note = Acetyl group
  BINDING 21: note = Amine group
KRIVQRIKDFLRAGPIKDFLR

SEQ ID NO: 6 - protein, 21 aa, synthetic construct
  BINDING 1: note = acetyl group
  BINDING 21: note = Amine group
KRIVQRIKDFLRAGPRRWWRW

SEQ ID NO: 7 - protein, 21 aa, synthetic construct
  BINDING 1: note = Acetyl group
  BINDING 21: note = Amine group
KRIVQRIKDFLRAGPRRLVRI